(19) United States
(12) Patent Application Publication
(10) Pub. No.: US 2024/0148627 A1
(43) Pub. Date: May 9, 2024

(54) GERMICIDAL FOAM FOR THE SKIN

(71) Applicants: Tzintzun CARRANZA LÓPEZ, Iztapalapa (MX); Luis Ariel CONTRERAS HUERTA, Iztapalapa (MX)

(72) Inventors: Tzintzun CARRANZA LÓPEZ, Iztapalapa (MX); Luis Ariel CONTRERAS HUERTA, Iztapalapa (MX)

(21) Appl. No.: 18/264,042

(22) PCT Filed: Feb. 5, 2021
(86) PCT No.: PCT/MX2021/050007

§ 371 Date: Aug. 2, 2023

(51) Int. Cl. A61K 8/41; A61K 8/04; A61K 8/19; A61K 8/34; A61K 8/73; A61K 8/81; A61Q 17/00

(52) U.S. Cl. CPC A61K 8/416; A61K 8/046; A61K 8/19; A61K 8/345; A61K 8/41; A61K 8/731; A61K 8/817; A61Q 17/005

(57) ABSTRACT

A composition of a germicidal solution in the form of foam, which prevents microbial contamination by bacteria, fungi and lipid-enveloped viruses, without irritating the user's skin is disclosed. The foam composition includes ammonium quaternary salts, which have a disinfectant effect against bacteria, fungi and certain types of virus; and hydroxyethyl cellulose, propylene glycol, polyvinylpyrrolidone K30, boric acid, amine oxide, and other pharmaceutically acceptable excipients or additives, such as emollients, humectants, solar protectors and UV protectors in pharmaceutically effective amounts.

### TECHNICAL FIELD

The present invention is directed to the field of broad-spectrum dermal disinfectants for local application. Particularly to foam that prevent microbial contamination from bacteria, fungi and viruses.

### BACKGROUND

Antibacterial, antiseptic or hand sanitizing agents make our lives easier in this hygiene-obsessed period. They are seen everywhere: in public bathrooms, in hospitals, in gyms, at the entrance and exit of places with a lot of human traffic, installed in easy operated dispensers. In addition, they are easy to carry in the pocket in small plastic bottles. A few drops are enough and ready because the hands are considered to be clean and disinfected. However, a research made by the US Centers for Disease Control and Prevention (CDC) indicates that hand sanitizers and antibacteriales are not as effective against some types of germs. Although the CDC notes that in most cases hand sanitizing with these bottled agents works just as well as washing with soap and water, there is a limit, these products are limited in eradicating all germs and in the conditions of effectiveness. For example, Staphylococcus aureus causes skin infections and sometimes pneumonia, and is present in hospitals and other healthcare settings, alcohol-based hand sanitizer gels can be effective in killing these micro-organisms, but these must contain at least 60% of alcohol to clean hands well. Non-alcoholic or low-alcohol gels are ineffective: they do not work against all types of germs on the hands, particularly norovirus, one of the causes of gastroenteritis.

Use contraindication of disinfectants is that antibacterial products with high-alcohol content can cause poisoning if are ingested, particularly by children. In addition, the CDC cautions that while many antibacterial products can reduce the number of germs on hands in some situations, they do not eliminate them entirely. The result is that these bacteria can develop a resistance.

There are also situations where antibacterial agents do not work very well, such as when your hands are too much oiled or dirty. These conditions can occur when camping, practicing a sport or gardening.

Antibacterial products are also not very good at removing harmful chemicals from your hands, such as pesticides and heavy metals.

Some hand products do not protect against salmonella, the E. coli, antibiotic-resistant Staphylococcus aureus (MRSA), and the norovirus, which can cause serious complications, including death. These four diseases can be transmitted from person to person. In the case of salmonella and E. coli, cause diarrhea that can be fatal in severe cases. Contagion can happen by contact with feces or with food that has not been properly refrigerated.

Norovirus is a common illness that is often contracted on cruise ships and is easily transmitted between passengers and crew. It is the main cause of the symptoms of gastroenteritis or “intestinal flu” and antibacterial products simply cannot kill it. In the case of the coronavirus, it can be effective, but only as a temporary measure, while you have access to soap and water.

The patent application MX/a/2016/005243; ORGANIC BIODEGRADABLE AND ANTIBACTERIAL HAND AND SKIN GEL describes an organic biodegradable and antibacterial hand and skin gel, characterized by being made from carbomer, triethanolamine, glycerin and chitosan, which does not affect the environment or cause irritation to the skin or in hands.

In the application MX/A/2009/007194; CRYSTALLINE ANTIBACTERIAL GEL WITH CITRUS AROMA WITH MOISTURIZERS AND METHOD FOR OBTAINING IT, a composition of crystalline antibacterial gel for hands with citrus fragrance with moisturizing characteristics is provided, which is mainly used in places where water is scarce; for daily use several times a day, and suitable for children. This is accomplished by adding natural humectants and using an antibacterial disinfectant agent, pure cane ethylic alcohol. The composition consists of different compounds that include: rheological modifier, sequestrant, fragrance, emulsifier, neutralizer, humectants, solvent, and disinfectant.

The application PA/A/2006/014962, ANTIBACTERIAL AND ANTICORROSIVE COMPOSITION FOR SKIN PROTECTION, provides an aqueous emulation comprising from 50% to 85.0% of water, from 0.5% to 5.0% of other ingredients such as the active ingredient, the active ingredient includes but is not limited to oil-soluble vitamins, water-soluble vitamins, active fatty acids, fatty acids, fatty alcohol esters of fatty acids, hydrocarbons, liquid and solid waxes, and silicone liquids of various kinds, the composition can also include preservatives and fragrance oils, the compositions can also include essential oils, with a layer that applied to the skin will remain insoluble in water, this layer protects the skin when it dries, even against friction. The composition can be applied to the skin in different pharmaceuticals forms; these application forms include but are not limited to gels, creams, lotions, sprays, ointments, and aerosols.

An alternative solution is antiseptic foams, which are based on antiseptic alcohol solutions. Antiseptic alcohol solutions serve to reduce the number of viable microorganisms on hands, reducing the risk of spreading infectious diseases.

These products contain antiseptic agents or antimicrobial substances, including alcohols, chlorhexidine, chlorine, hexachlorophene, iodine, chloroxylenol (PCMX), quaternary ammonium compounds, and triclosan.

Although, in principle, in terms of the effectiveness of the composition, they may be similarities with the antibacterial gel, but the difference is the liquid or foam physical form. In the first place, if we take a liquid solution, it is easy to spill out half of the liquid that is poured, for that reason its effectiveness will be diminished; the hydro-alcoholic foam solution finishes the above problems, even with a smaller amount of product, which means less overall consumption.

The hydro-alcoholic solution in foam is more effective, since it is achieved that the entire dose of product applied stays in the hands without spilling, its distribution is homogeneous, and less quantity is needed to achieve the same effectiveness as in liquid or gel.

Regarding the effectiveness of hydro-alcoholic solutions in particular, they eliminate 99.99% of the most common germs, protect and hydrate the skin.

From around 2004 and 2006, when avian flu spread worldwide, there was a boom in the creation of companies and products of this type, without any type of certification and without quality guarantees. Keep in mind that antiseptic agents tend to dry out and attack the skin, if they are not in the right combination and quantity, without forgetting that using this type of product must guarantee the care and protection of the skin, not only for aesthetic purposes, but not because otherwise the natural protective layer of the skin can be progressively eliminated.

### DETAILED DESCRIPTION OF THE INVENTION

The object of the present invention is providing a composition of an antiseptic solution in the form of foam, wherein the foam is germicidal, disinfectant and does not irritate the skin against: the foam composition is effective against bacteria, fungi and lipid-enveloped viruses. Its disinfectant and protective action have a lasting effect. It contains quaternary ammonium salts that have a disinfectant effect against bacteria, fungi and some types of viruses.

Said germicidal foam is not toxic, flammable or destructive to the ozone layer. In addition, it protects the skin from contamination by microorganisms. The germicidal foam has a pH between 6 and 7, it is completely soluble in water; whose density at 25° C. is 1 g/ml; and is stable under normal conditions of storage and use for up to 24 months.

The germicidal foam is administered after washing the area where the product will be applied with soap and water, rinsing and drying completely. The antiseptic foam is applied, distributing evenly throughout the area; when applying to hands, it must be ensured that the foam comes into contact with the cuticle and the skin under the nails; let it air dry. This procedure can be repeated again after 4 hours.

The foam object of the present invention avoids cross contamination for 4 hours; it contains quaternary ammonium salts that have disinfectant activity against bacteria (mainly Gram positive), fungi and viruses, thus helping to prevent infections caused by these microorganisms, it is not irritating to the skin and does not cause cracking or irritation to the skin. The antiseptic foam is not toxic and is not absorbed, so it does not impede the breathing of the pores or decrease the sensitivity; it is free of dyes, fragrances, alcohol and silicone.

The composition of the antiseptic solution to obtain the antiseptic foam object of the present invention comprises alkyl dimethyl benzyl ammonium hydrochloride between 0.1 and 1.0% w/v, hydroxyethyl cellulose from 0.05 to 2.5% w/v, propylene glycol from 2.0 to 4.0% w/v, Polyvinylpyrrolidone K30 from 0.5 to 3.0% w/v, boric acid 0.05 to 1.0% w/v, amine oxide 1.0 to 3.5% w/v, and purified water; it can also comprise other pharmaceutically acceptable excipients or additives, such as emollients, humectants, sunscreens, UV protectors, among others in pharmaceutically effective amounts.

### Example of Preparation

The composition of the germicidal foam of the invention is prepared according to the following procedure:

In a stainless steel mixing tank containing 1.8 liters of purified water, 60 kg of propylene glycol are added; it is stirred until the solution is homogeneous for approximately 15 minutes. 2 kg of hydroxyethylcellulose is added to the previous mixture and it is mixed until homogeneous for approximately 1 hour at 560 rpm; once the homogenization period is over, 60 kg of Polyvinylpyrrolidone K30 are added and shake until completely dissolved for 30 minutes, then 1.2 kg of boric acid are added, mixing for 15 minutes. When the above solution is homogeneous, 6.5 kg of alkyl dimethyl benzyl ammonium hydrochloride are added, the mixture is kept under stirring for 30 minutes and 40 liters of amine oxide are added and the mixture is homogenized. Add water to a volume of 200 liters and keep stirring for 15 minutes. Drain the tank by filtering the obtained solution. Pack in foam-producing containers similar to commercially available devices.

### Clinical Trials

The composition of the germicidal foam obtained according to the procedure described above was subjected to various clinical tests, such as: Evaluation of the bactericidal activity according to the NF standard EN 13727+A2:2015; Skin tests to assess the potential for skin irritation after contact with a cosmetic product; Evaluation of the activity in yeast (fungi) according to the NF standard EN 13624: 2013 and Evaluation of the virucidal activity according to the NF standard EN 14476+A2: 2019.

### Evaluation of the Bactericidal Activity According to the NF Standard EN 13727+A2:2015.

This assay was carried out by LABORATORIE MIDAC, France.

The foam object of the present invention was tested in separate culture media each containing a separate pair: Pseudomonas aeruginosa DSM939; Staphylococcus aureus DSM 699; Eterococcus hirae DSM 3320 and Escherichia coli K12 Dsm 11250, incubated at a temperature of 37° C. (±1° C.). The test was carried out at a temperature of 20° C. (±1° C.); with a contact time of 30 seconds (±5 sec) and 0.3 g/l of bovine albumin (clean conditions) as an interfering substance.

Table of results 1Reduction ® of the number of
viable cells in the tested concentration (VN):

| Strain | 1% | 50% | 80% |
|---|---|---|---|
| Pseudomonas aeruginosa | Log R: 3.56 | Log R: >5.33 | Log R: >5.33 |
| DSM939 | | | |
| Staphylococcus aureus | Log R: 4.57 | Log R: >5.52 | Log R: >5.52 |
| DSM 699 | | | |
| Eterococcus hirae | Log R: 4.60 | Log R: >5.50 | Log R: >5.50 |
| DSM 3320 | | | |
| Escherichia coli K12 | Log R: 4.60 | Log R: >5.44 | Log R: >5.44 |
| Dsm 11250 | | | |

Active concentration if R ≥ 5
Non-active concentration if R < 5

According to the NF standard EN 13727: 2015, the germicidal foam object of the present invention has bactericidal activity at a concentration of 50% after 30 seconds (±5 sec) at 20° C. (±1° C.) in contact with 0.3 g/l bovine albumin (clean conditions).

Skin Tests to Evaluate the Potential of Irritation Skin after Contact with a Cosmetic Product.

This trial was carried out by COMPLIFE Italia S.r.l.

An 8 mm diameter “Finn Chamber®” aluminum disc containing a blotting paper disc moistened with a sample of the product to be tested was fixed to the skin of ten (10) volunteers with a tape that has been tested for its safety and that ensures the occlusive application of the product. A sufficient amount was applied to saturate the pad but not overflow it when applied to the skin. The product was left in contact with the skin surface for 48 hours. Skin reactions were analyzed at 15 minutes, one hour and 24 hours after the Finn Chamber® was removed. A Finn Chamber® containing the blotting paper disc soaked in mineral water was applied and used as a negative control.

The skin reaction measurement scale used is shown below:

TABLE 1
Clinical skin reaction scale

| | |
|---|---|
| No Erythema | 0 |
| Light erythema (hardly visible) | 1 |
| Clearly visible erythema | 2 |
| Moderate erythema | 3 |
| Severe erythema (dark red with possible slight scarring) | 4 |
| No Edema | 0 |
| Light edema (hardly visible) | 1 |
| Light edema | 2 |
| Moderate edema (approximately 1 mm of raised skin) | 3 |
| Severe edema (swelling extending even beyond the | 4 |
| area of application) | |

TABLE 2
Classification of the average irritation index (according
to the amended Draize classification)

| Average Irritation Index ( | Product Classification |
|---|---|
| <0.5 | Non-irritating |
| 0.5 ≤ IIM < 2.0 | Slightly irritating |
| 0.5 ≤ IIM < 5.0 | Moderately irritating |
| 0.5 ≤ IIM ≤ 8.0 | Highly irritating |

The results obtained are shown below:

Mean values for edema (Ed) and erythema (Er)

| IIM | IIEM | IIM | IIM | IIM | IIEM |
|---|---|---|---|---|---|
| Er 15′ | Ed 15′ | Er 1 hr | Ed 1 hr | Er 24 hr | Ed 24 hr |
| 0.10 | 0.00 | 0.10 | 0.00 | 0.10 | 0.00 |

Average values of the irritation index

| IIM 15′ | IIM 1 hr | IIM 24 hr |
|---|---|---|
| 0.10 | 0.10 | 0.10 |

The tables shown above contain the values of the edema and erythema indices recorded for the volunteers. The irritation potential of the product was evaluated according to the amended Draize classification. Based on the results obtained, it is evident that the product does not cause any irritation to the skin.

### Evaluation of Fungicidal Activity According to the NF Standard EN 13624:2013.

It was carried out by LABORATORIE MIDAC, France.

The foam object of the present invention was tested in a culture medium for Candida albicans DSM 1386, incubated at a temperature of 30° C. (±1° C.). The trial was carried out at a temperature of 20° C. (±1° C.); with a contact time of 30 seconds (±5 seq) and 0.3 g/l of bovine albumin (clean conditions) as interfering substance.

Table of results 1Reduction ® of the number of
viable cells in the tested concentration (V/V):

| Strain | 1% | 50% | 80% |
|---|---|---|---|
| Candida albicans | Log R: <1.93 | Log R: >3.77 | Log R: >4.25 |
| DSM1386 | | | |

Interpretation criteria:
Active concentration If R ≥ 4
Non-active concentration if R < 4

According to the NF standard EN 13624:2013, the product has fungicidal activity at a concentration of 80% after 30 seconds (±5 sec) at 20° C. (±1°) in contact with 0.3 g/l of bovine albumin (clean conditions).

### Evaluation of the Virucidal Activity According to the NF Standard EN 14476+A2:2019.

This trial was carried out by LABORATORIE MIDAC, France.

The composition of the foam object of the present invention was tested in a culture medium for Vaccina virus strain p1, Elstree activated, incubated at a temperature of 30° C. (±1° C.), where the used method for the inactivation of the product was used with a Microspin S-400 HR column, according to the manufacturing protocol, the identification of the cells was Vero cells, CCL-81, p6, ME 10% SVF, 1% NAAE, 1% ATB, 1% L-Glu. The trial was carried out at a temperature of 20° C. (±1° C.); with a contact time of 60 seconds (±5 sec) and 0.3 g/l of bovine albumin (clean conditions) as an interfering substance.

### Trial Method

One part of the interference substance, one part of the virus suspension and eight parts of the biocide agent were mixed; the mixture was incubated at the indicated temperature and for the established contact time. The assays were validated by a cytotoxicity control, an interference control, a neutralization control, and an internal formaldehyde standard. The reduction titer calculation is based on the Spearman and Kärber method and it is measured as the difference between the virus control titer and the test product solution titer.

Results:
Virus suspension: 8.00E+00 log DIT C₅₀.
Maximum detectable virus inactivation: 5.50 E+00 log DIT C₅₀.
Reference trial inactivation of the virus inactivation after 15 minutes: 3.13E+00 log DIT C₅₀.

Viricide study:

| | | Viral titer | |
|---|---|---|---|
| Product | Interfering | in the trial | Reduction |
| concentration | substance | (log DIT C₅₀.) | (log DIT C₅₀) |
| 1% v/v | 0.3 g/l bovine albumin | 4.00E+00 | 4.00E+00 |
| 50% v/v | 0.3 g/l bovine albumin | 2.50E+00 | 5.50E+00 |
| 80% v/v | 0.3 g/l bovine albumin | 2.50E+00 | 5.50E+00 |
| Viral control | | 7.63E+00 | |
| TO | | | |
| Viral control | | 8.00E+00 | |
| Tmax | | | |

In accordance with the NF standard EN 14476+A2:2019, the product has virucidal activity at a 50% concentration after 60 seconds (±5 sec) at 20° C. (±1° C.) in contact with 0.3 g/l of bovine albumin (clean conditions) against Vaccinia virus Elstree strain.

1. A composition of antiseptic solution to obtain a germicidal foam, comprising: alkyl dimethyl benzyl ammonium between 0.1 and 1.0% w/v, hydroxyethyl cellulose from 0.05 to 2.5% w/v, propylene glycol from 2.0 to 4.0% w/v, Polyvinylpyrrolidone K30 from 0.5 to 3.0% w/v, boric acid 0.05 to 1.0% w/v, amine oxide 1.0 to 3.5% w/v, and purified water; emollients, humectants, sunscreens, UV protectors, among others in pharmaceutically effective amounts.

2. The composition of claim 1, characterized in that it comprises 0.13% w/v of alkyl dimethyl benzyl ammonium, 0.1% w/v of hydroxyethyl cellulose, 3.0% w/v of propylene glycol, 3.0% w/v of Polyvinylpyrrolidone K30, 0.6% w/v boric acid, 2.0% w/v amine oxide, and purified water.

3. The composition of claim 1, characterized in that it comprises 0.32% w/v of alkyl dimethyl benzyl ammonium, 0.1% v/v of hydroxyethyl cellulose, 3.0% w/v of propylene glycol, 3.0% w/v of Polyvinylpyrrolidone K30, 0.06% w/v boric acid, 2.0% w/v amine oxide, and purified water.

4. The composition according to claim 1, characterized in that it comprises quaternary ammonium salts that have a disinfectant effect against bacteria, fungi and lipid-enveloped viruses.

5. The composition according to claim 1, characterized in that it has a pH between 6 and 7, is soluble in water; with a density at 25° C. of 1 g/ml; and it is stable under normal conditions of storage and use for up to 24 months.

6. The composition according to claim 1, characterized in that it is a disinfectant, and has a protective action on the skin against: microorganisms, oils, greases, solvents, paints, alkalis, acids, etc.; where its disinfectant and protective action is effective for 4 hours.

7. A process for the preparation of the composition according tom claim 1, wherein said process consists of:
dissolve propylene glycol in purified water and homogenize the solution for approximately 15 minutes;
a) adding hydroxyethylcellulose and mixing for approximately 1 hour;
b) adding Polyvinylpyrrolidone K30 and stirring for 30 minutes;
c) adding boric acid while mixing for 15 minutes;
d) adding alkyl dimethyl benzyl ammonium hydrochloride while stirring for 30 minutes;
e) adding amine oxide and homogenizing the mixture;
f) adding water and keep stirring for 15 minutes; and
g) unloading the mixture from the tank by filtering the solution obtained and packaging it in foam-producing containers.
